(19) United States
(12) Patent Application Publication
(10) Pub. No.: US 2007/0207188 A1
(43) Pub. Date: Sep. 6, 2007

(54) Cocoa products and methods of treating cardiovascular conditions with sugar-free cocoa

(76) Inventors: Debra L. Miller, Palmyra, PA (US); David L. Katz, Hamden, CT (US)

Correspondence Address: WILEY REIN LLP, 1776 K. STREET N.W., WASHINGTON, DC, 20006, US

(21) Appl. No.: 11/707,279

(22) Filed: Feb. 16, 2007

Related U.S. Application Data
(60) Provisional application No. 60/777,708, filed on Feb. 16, 2006.

(51) Int. Cl. A61K 9/68; A61K 36/898

(52) U.S. Cl. 424/440; 424/776; 426/660

(57) ABSTRACT

The invention provides, in one aspect, a method of administering a product comprising a cacao solids-containing, cocoa powder or extract containing cocoa polyphenols to improve endothelial function. The sugar-free compositions and methods of using them exhibit synergistically improved cardiovascular benefits compared to other compositions. Preferred sugar-free chocolate products and cocoa beverages are also disclosed.

### REFERENCE TO RELATED APPLICATIONS

This application claims full priority benefit of prior U.S. Provisional application No. 60/777,708, filed Feb. 16, 2006, the entire contents of which are hereby incorporated by reference.

### FIELD OF THE INVENTION AND INTRODUCTION

The invention relates to sugar-free cocoa-containing products, cocoa polyphenol-containing products, related products and their use in treatments for reducing cardiovascular risk factors and improving health. A variety of compositions for human or animal consumption or use are described as well as treatment regimens for improving cardiovascular function, cardiovascular health, and overall health. In one embodiment, the sugar-free cocoa-containing products can also be low fat or fat-free products, and/or contain additional food additives or supplements. These products can be used, inter alia, in methods and/or a regimen for health maintenance, improvement of various cardiovascular or vascular conditions, and/or for weight loss.

### RELEVANCE OF THE INVENTION AND DESCRIPTION OF RELATED ART

Heart disease, and more generally cardiovascular disease, is the leading cause of death in the U.S. for those aged 35 and older. Much research has been devoted to identifying quantifiable risk indicators that can predict the onset of cardiovascular diseases. A technique known in general as endothelial function testing represents a growing and powerful measure of the overall susceptibility to cardiac events. Vascular endothelial function is a measure of blood vessel behavior. When blood vessels dilate or constrict properly, endothelial function is considered to be normal. The reactivity of blood vessels to variations in blood flow is an indicator of heart health. Specifically, abnormal vascular endothelial function has been recognized as an early marker of cardiovascular disease. See Bugiardini, et. al., Endothelial Function Predicts Future Development of Coronary Artery Disease, Circulation 109:2518-2523 (2004).

Flow mediated dilation (FMD) is a non-invasive method of studying endothelial function. See Celermajer D. S., et. al., Non-invasive detection of endothelial dysfunction in children and adults at risk of atherosclerosis, Lancet 340:1111-1115 (1992). FMD measures the increase in arterial diameter in response to an increase in blood velocity following a period of cuff-induced ischemia. The technique is traditionally employed in the brachial artery of the arm.

The brachial artery has been noted for its similarity in function to coronary arteries.

Thus, the brachial artery is considered a superb and accessible vessel as well as a non-invasive and predictive model for the status of coronary arteries, such that the FMD assays of the brachial artery described herein, and thus are an excellent aid in predicting future cardiovascular complications. See Schroeder S, et. al., Noninvasive determination of endothelium-mediated vasodilation as a screening test for coronary artery disease: pilot study to assess the predictive value in comparison with angina pectoris, exercise electrocardiography, and myocardial perfusion imaging, Am Heart J. 138(4 Pt 1):731-9 (1999).

Various investigations show a relationship between cocoa polyphenols and endothelial function. In fact, a number of dietary compounds with antioxidant activity have been suggested to have pronounced effects on endothelial function. See, for example, Vita, J. A., Polyphenols and cardiovascular disease: effects on endothelial and platelet function, Am J Clin Nutr 81:292S-7S (2005). Two studies using dark chocolate or cocoa found significant improvement in the overall function of the endothelium in healthy volunteers. See Engler M B, et al., Flavonoid-rich chocolate improves endothelial function and increases plasma epicatechin concentrations in healthy adults, J Am Coll Nutr 2004 23: 197-204; Heiss C, et al., Vascular effects of cocoa rich in flavan-3-ols, JAMA 2003 290:1030-1. Another study investigated this effect in participants with high blood pressure and found 100 g of dark chocolate each day for 15 days produces significant improvements in endothelial function as well as other health factors related to insulin resistance and blood pressure. See Grassi D, et al., Cocoa reduces blood pressure and insulin resistance and improves endothelium-dependent vasodilation in hypertensives, Hypertension 2005 46: 1-8.

While these studies show cocoa flavanols or polyphenols can have a potential health benefit, none even suggest the potential synergistic effect of consuming cocoa with other dietary compositions or in a particular cocoa composition or formulation. As shown in detail below and in one aspect, the present invention provides new compositions and novel uses for sugar-free formulations of cocoa that have demonstrable and previously undisclosed improved characteristics in endothelial function when compared to conventional cocoa and chocolate compositions with nutritive sugars. In another aspect, the invention makes a novel use of the previously unknown synergistic effect of administering cocoa, or cocoa polyphenols, in the absence of sugar on vascular endothelial function. The improvements seen in subjects that take sugar-free cocoa can be compared to the use of dark chocolate compositions administered to similar groups of subjects. The improved effect for sugar-free compositions can also be used in, or incorporated into, other new or existing food products and dietary regimen, so that, for example, low fat or fat-free products can be produced and used to create a synergistic and beneficial health effect with cocoa powders, extracts, and cocoa polyphenols.

### BRIEF SUMMARY OF THE INVENTION

The present invention relates to compositions for and methods of improving endothelial function using cocoa with nutritive sugars substantially absent from the formulation. More generally, the compositions and methods can be used as prophylactic or therapeutic treatments to improve cardiovascular conditions and/or prevent cardiovascular diseases, for example but not limited to treating symptoms or aspects of atherosclerosis, thrombosis, restenosis, hypertension, heart attack, stroke, or vascular circulation conditions, and/or improvements in risks factors associated with any of these diseases or conditions, or similar or related diseases or conditions. The characteristics and/or improvements in risk factors can be assessed by any of a number of available or known methods, including, for example, the FMD procedures. Specifically and in one embodiment, the present invention relates to compositions containing natural cocoa with intact cocoa flavanols in combination with non-nutritive sweeteners for preventing, minimizing or reversing deleterious cardiovascular conditions or disease states.

As shown below, a randomized, placebo-controlled, clinical study of human endothelial function demonstrates that sugar-free cocoa has a remarkably strong effect on endothelial function status. Data from this study also indicate that the effect was surprisingly improved compared to the effects of cocoa with a nutritive sugar, where the cocoa product contents are controlled between the two groups. The sugar-free compositions have surprisingly more than six times the beneficial effect to that of a nutrient-matched, non-cocoa containing placebo, and over two times or over three times the beneficial effect compared to cocoa with sugar (sugar cocoa). The sugar-free cocoa preparations and compositions and methods of the invention improved endothelial function status much more robustly than oatmeal, soy foods, the antioxidant vitamins C and E, as well as well as other foods and dietary compounds (see Katz, D. L., et al., J Am Coll Nutr, 23(5):397-403 (2004); Katz, D. L., et al., Prev Med, 33(5):476-84 (2001)). However, and as noted above, the sugar-free compositions can be combined with other healthy ingredients, additives, or food products, such as, for example, antioxidants, soluble fiber, arginine, omega-3 fatty acids, oatmeal, soy, and foods with vitamin additives, such as vitamin A and C. The combinations formed can advantageously contain one or more sugar substitutes, such as non-nutritive sweeteners, sugar alcohols, polyols, and/or high intensity sweeteners, as known and used in the art, in new food products that retain the synergistic effects noted and demonstrated here.

Thus, in one aspect, the invention comprises a composition containing natural cocoa powder or cocoa extracts in combination with sugar substitutes, where the cocoa powder or extract represents more than about 60% or more than about 80% of the composition by dry weight, or the equivalent of about 85-95% of the composition by dry weight. Another aspect of the invention is a method for improving endothelial function in humans comprising administering natural cocoa powder and a sugar substitute, where the cocoa powder constitutes at least about 60% or at least about 80%, or at least 85-95% of the formulation by dry weight. As known in the art, natural cocoa is the product resulting from pressing the lipid content from fermented and/or roasted cocoa bean nibs. Approximately 10-25% of the lipid fraction (cocoa butter) is retained in natural cocoa. Furthermore, natural cocoa is not treated with alkali or similar agents used in cocoa processing, a process known as “Dutching.”

In particularly preferred embodiments, the invention comprises efficacious cocoa-containing beverages and methods of using them to improve cardiovascular conditions, endothelial function, or prevent cardiovascular disease. FIG. 2 shows the change in FMD two hours following ingestion of a sugar-free cocoa-containing beverage composition of the invention with a sugar substitute (for example, aspartame, Ace-K, and mixtures of them), compared to cocoa with nutritive sweetener (sugar, or more particularly sucrose) and a placebo containing no cocoa polyphenols. Thus, it is one aspect or object of the invention to provide a sugar-free composition of cocoa having a measurable efficacy in improving endothelial function and capable of being administered in sufficient concentration as an easy to consume product, such as a beverage, bar, confectionary product, pill, tablet, capsule, liquid or ingredient in a food product or mix. Certain embodiments of the invention can be made into individualized packets or sachets produced and sized to accommodate a mix or composition for producing a beverage when placed in water or other liquid. A number of other consumable products and mixes can be devised by one of ordinary skill in the art.

Another aspect of the invention is to provide a composition of sugar-free cocoa in combination with one or more non-nutritive sweetening agents so as to produce a pleasant tasting cocoa product while maintaining the synergistic effect provided by the cocoa polyphenols in the absence of sugar. Another aspect of the invention is to provide a composition of sugar-free cocoa in combination with other available or known food products, ingredients, or agents, such as those known to cause vasodilation, or those known to improve vascular conditions. Green tea, green tea extracts, blueberry or blueberry extracts, red or white wine products, grape extracts, and other foods and ingredients having polyphenolic compounds may be considered and used in these combinations. Yet another aspect of the invention is to provide a composition of sugar-free cocoa in combination with other available or known supplements, active ingredients, appetite suppressing agents, and similar agents or ingredients, especially those also found in antioxidant-containing foods, herbs, or food product ingredients. Further, the composition may also or in addition be combined with one or more other dietary nutrients, such as vitamins, minerals, amino acids, etc., to provide a nutritional supplement. Any of these combinations of the invention can advantageously provide benefits to human health and capture additional beneficial effects, such as reducing caloric intake, on cardiovascular and overall health. As noted above, all of the ingredients or combinations may be combined with appropriate binders, preservatives and other edible or ingestible compounds known to one of skill in the art in the industry to produce a suitable pill, tablet, capsule, liquid or ingredients in a food including, health bars and beverages.

A further aspect of the invention is to provide a treatment regimen that optimizes both the quantity and frequency of sugar-free cocoa consumption to achieve an acceptable or measurable efficacy in the improvement of endothelial function. As one example of this embodiment, a treatment regimen where about 10 to about 20 grams, or about 20 to about 30 grams, or about 30 to about 40 grams, or about 40 to about 50 grams of non-alkalized cocoa powder are administered once or twice a day without nutritive sugar or without sucrose. In addition, embodiments of the invention can incorporate sweeteners, such as sugar alcohols, that are absorbed or active slower in comparison to glucose. Similarly, the compositions and methods can incorporate foods or ingredients with a relatively low or low glycemic index, such as a GI of about 70 or below, or at or below about 60, or at or below about 50, or at or below about 40, or at or below about 30.

Throughout this disclosure, applicants refer to journal articles, patent documents, published references, web pages, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources. Section headings within this document should not be taken as an admission of the relevance of any particular document or cited work and are presented merely for organizational purposes. The description and examples that follow are merely exemplary of the scope of this invention and content of this disclosure and do not limit the scope of the invention. In fact, one skilled in the art can devise and construct numerous modifications to the examples listed below without departing from the scope of this invention.

### BRIEF DESCRIPTION OF THE DRAWINGS

FIG. 1 depicts a graph of changes in blood pressure following consumption of various compositions during an acute, 7 day test period. Changes in blood pressure are measured along the Y-axis as expressed in mmHg. Changes in both systolic blood pressure (SBP) and diastolic blood pressure (DPB) are depicted in separate bars for each of three compositions. The three compositions consist of a placebo, a cocoa+sugar composition and a sugar-free composition of the invention. Decreases in blood pressure are considered favorable results, while increases in blood pressure are considered unfavorable.

FIG. 2 depicts a graph of changes in flow mediated dilation (FMD) following consumption of the same three compositions depicted in FIG. 1. Changes in FMD are depicted as a percentage along the Y-axis with increases in FMD considered favorable results and decreases in FMD being unfavorable.

FIG. 3 depicts a graph of changes in stimulus adjusted response measure (SARM) along the Y-axis for the same three compositions depicted in FIGS. 2 and 3. SARM is defined as flow mediated dilation (FMD) divided by the change in blood flow through the vessel 15 seconds after cuff-induced ischemia is terminated. Increases in SARM are considered favorable results and decreases in SARM are unfavorable.

FIG. 4 depicts a graph of changes in blood pressure following consumption of dark chocolate compared to white chocolate control during an acute, 7 day test period. Changes in blood pressure are measured along the Y-axis as expressed in mmHg. Changes in both systolic blood pressure (SBP) and diastolic blood pressure (DPB) are depicted in separate bars. Decreases in blood pressure are considered favorable results, while increases in blood pressure are considered unfavorable.

FIG. 5 depicts a graph of changes in flow mediated dilation (FMD) following consumption of the same chocolate compositions in FIG. 4. Changes in FMD are depicted as a percentage along the Y-axis, with increases in FMD considered favorable results and decreases in FMD being unfavorable.

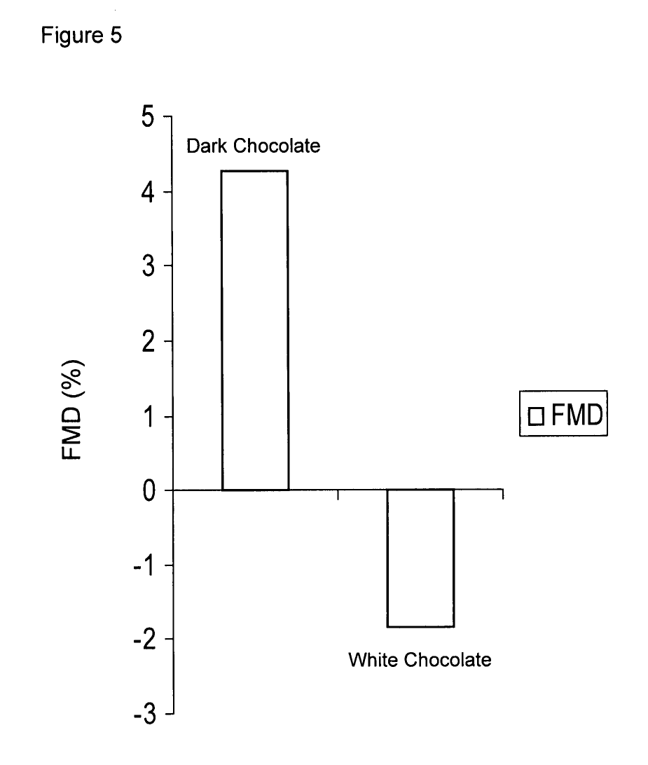

FIG. 6 depicts a graph of changes in stimulus adjusted response measure (SARM) along the Y-axis for the same chocolate compositions in FIGS. 4 and 5. SARM is defined as flow mediated dilation (FMD) divided by the change in blood flow through the vessel 15 seconds after cuff-induced ischemia is terminated. Increases in SARM are considered favorable results and decreases in SARM are unfavorable.

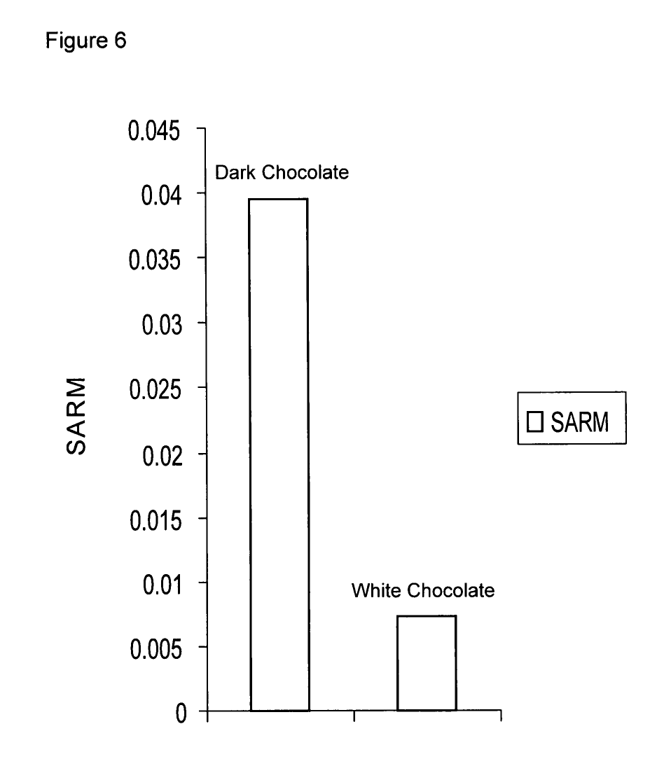

### DETAILED DESCRIPTION OF EXEMPLARY EMBODIMENTS

In general and as used in this invention, the various chocolate and cocoa-containing products and compositions noted here refer to the terms as used in Minifie (Chocolate, Cocoa, and Confectionery, 3d ed., Aspen Publishers), specifically incorporated herein by reference. The cacao bean refers to the cacao bean, also called cocoa bean, in nature and a cocoa containing product is a product derived from or having some component derived from the cocoa bean. The nib refers to the cacao bean without the shell and is approximately 54% fat and 46% non fat solids on a dry basis. Non fat cocoa solids are the processed non fat solids of chocolate liquor. Cocoa powder refers typically to cocoa solids with a total of 10% to 25% fat, or more typically 10% to 12% fat, where the fat is generally cocoa butter. In addition, cocoa powder can also be processed from extraction methods, where substantially all of the fat is removed. The natural cocoa and natural cocoa powders as used here can be any of these cocoa powders as long as they are not dutched cocoa, as noted below. The cocoa polyphenol compositions of the invention generally contain natural cocoa or cocoa powder, which is the product resulting from pressing the lipid content from fermented and roasted cacao bean nibs. Approximately 10-25% of the lipid fraction (cocoa butter) is retained in natural cocoa. Furthermore, natural cocoa and cocoa powder is not treated with alkali or other agents known to the cocoa processing industry for use in a process known as “Dutching,” unless it is specifically indicated as dutched, alkalized, or dutch process cocoa. Breakfast cocoa is cocoa solids with 20 to 24% fat, where the fat is generally cocoa butter. Chocolate liquor (or cocoa liquor) is ground cacao bean nibs and it can be separated into cocoa butter and cocoa solids. Cocoa butter is the fat component of chocolate liquor, whereas the remaining part of chocolate liquor is cocoa solids or the cocoa mass. As one of skill in the art understands, a certain amount or percentage of cocoa solids in a food ingredient can be achieved, inter alia, by using or adding an amount of cocoa powder, chocolate liquor, or other chocolate or cocoa ingredient containing the requisite amount of cocoa solids. Similarly, a certain amount or Percentage of natural cocoa in a food ingredient can be achieved, inter alia, by using or adding an amount of cocoa powder, chocolate liquor or other chocolate or cocoa ingredient, including extracts of cocoa containing the active compounds known to cause vasodilation. Furthermore, while many different countries, including the U.S., specifically define food products containing cocoa or cocoa products as having certain ranges or ingredients, the terms chocolate, milk chocolate, and dark chocolate, are as used commonly in the US food industry and do not imply, unless stated otherwise, a specific content. In addition, while a cocoa containing product having a particular anti-oxidant or polyphenol level is not required, the invention encompasses the use of cocoa containing products with enhanced, altered, or increased levels of anti-oxidants or polyphenol compounds as compared to conventional cocoa containing products. A variety of production methods, extraction methods, and the addition of extracts or cocoa-derived polyphenols have been mentioned and can be used by one of ordinary skill in the art to create cocoa compositions of the invention. Other nutritional, therapeutic, or preventative ingredients can be added as well, as known in the art.

With respect to cocoa polyphenols, numerous studies have indicated a strong inverse correlation between heart disease and the consumption of polyphenol-containing compounds (Vita, J. A., Am J Clin Nutr 81(Suppl):292S-7S (2005)). Although cocoa beans and the products thereof have been considered to have health promoting properties for thousands of years—dating back to ancient Central American cultures, recent analytical technologies have allowed for characterization of specific chemistry of the cocoa bean (Hurst, W. J. et al., Nature 418: 289-90 (2002)). Analysis of cocoa, the main product of the cacao bean and the base material for chocolate and numerous baked goods, indicates that it is highly concentrated in certain polyphenol compounds, specifically in the flavanoid family. The most abundant flavanoids in cocoa are the flavanols, including the monomeric forms of epicatechin and catechin, as well as the oligomeric forms, called procyanidins. The monomeric forms as well as the lower level polymers have been demonstrated to be bioavailable, reaching peak plasma concentrations two hours post-consumption (Baba, S., Free Radic Res 33(5):635-412 (2000)).

Because endothelial dysfunction correlates strongly with cardiovascular disease and can be reversed in response to efforts to reduce risk (e.g., improved nutrition or exercise behavior), this biomarker can be chosen as one to test the effects of the current invention. Endothelial function refers to arterial vasomotor responses mediated predominantly by the release of nitric oxide (vasodilating) and endothelin (vasoconstricting) from the vascular endothelium, and plays an important role in the pathogenesis of atherosclerosis, hypertension, cardiovascular disease, stroke, and diabetes. There are many other markers, including serum markers, of cardiovascular risk, such as, but not limited to, low density lipoproteins (LDL), high density lipoproteins(HDL), total cholesterol, triglycerides, lipoprotein A, fibrinogen, antioxidant levels, apolipoproteins, leukocyte count, C-reactive protein, homocysteine. Any of these markers can be used to evaluate or measure the effect of the compositions or methods of the invention. However, endothelial function assessment may represent a superior marker of the overall effect of a treatment, intervention or exposure on the susceptibility to cardiac events and it is the preferred method of measuring or demonstrating cardiovascular or health benefits. As a measure of the vascular physiology that is influenced by the full range of circulating chemical mediators, endothelial function testing shows great promise as a single, non-invasive means of gauging the aggregate impact of dietary manipulations.

Endothelial function can be assessed non-invasively in the peripheral circulation by monitoring the vasodilation induced by increased blood flow shear stress (flow mediated dilatation—FMD). High frequency ultrasonographic imaging of the brachial artery to assess endothelium-dependent flow mediated vasodilatation (FMD) is a standard technique (Katz et al., Int'l J. of Cardiology 22 (2005); 65-70, incorporated herein by reference). Prior clinical studies of endothelial function assessed either in the coronary or brachial circulation have shown that FMD predicts subsequent cardiovascular risk. For example, individuals with cardiac risk factors are significantly more likely to have FMD<10% as compared to healthy counterparts.

The combinations, compositions, and products of the invention can be produced from a variety of cocoa products available or known. For example, a cocoa extract or cocoa powder can be used. The extract or a compound therefrom will contain cocoa procyanidin(s) and may be processed in a way to preserve or increase the concentration of polyphenols, flavanols, or other compounds. For example, a cocoa extract or compounds therefrom can be obtained by a process comprising reducing cacao beans to powder, defatting the powder, and optionally extracting and purifying certain compounds or active compound(s) from the powder. The extract or powder can be prepared by freeze-drying the cacao beans and pulp, depulping and dehulling the freeze-dried beans, and grinding the dehulled beans. An extraction step can optionally be used, where certain compounds can be removed or separated by solvent extraction techniques. In addition, extracts can be purified by gel permeation chromatography, preparative High Performance Liquid Chromatography (HPLC) techniques, or other techniques know to one of skill in the art.

In addition to the effects on endothelial function, the compositions and methods of the invention may also be useful in treating hypertension. Hypertension is a condition where the pressure of blood within the blood vessels is higher than normal as it circulates through the body. When the systolic pressure exceeds 150 mm Hg or the diastolic pressure exceeds 90 mm Hg for a sustained period of time, damage is done to body tissue, especially the kidney. For example, excessive systolic pressure can rupture blood vessels anywhere. When rupture occurs within the brain, an aneurysm results. It can also cause thickening and narrowing of the blood vessels which can lead to atherosclerosis. Elevated blood pressure can also force the heart muscle to enlarge as it works harder to overcome the elevated resting (diastolic) pressure when blood is expelled. Measuring effects on hypertension or blood pressure are routine in the art, and any available or known technique can be adopted.

As mentioned above, various dietary additives, supplements, minerals, and vitamins, and herbal and ingestible extracts can be added to the cocoa formulations or compositions of the invention, or used in the methods. For those employing an appetite suppressant, in general, these compounds or compositions or extracts decrease the desire for food for a period of time. Commercially available appetite suppressants include, but are not limited to, amfepramone (diethylpropion), phentermine, mazindol and phenylpropanolamine fenfluramine, dexfenfluramine, and fluoxetine. Various peptide and polypeptide compounds have also bee tested and/or suggested for this purpose, including, but not limited to, Neuropeptide Y, PYY, pseudo-peptides, CCK, and fragments thereof. Various plant and herbal extracts have been tested and suggested also, including, but not limited to epigallocatechin gallate from green tea extracts and green tea extracts themselves; theobromine and high doses of theobromine, “high” meaning over 250 mg. per day, preferably over 1000 mg. per day; and cinnamon bark extracts.

Additional food ingredients or edible ingredients can be combined with any of the compositions and combinations of the invention. Especially preferred are one or more of those prepared as USP ingredients (United States Pharmacopeia standard) and those known as the GRAS (generally recognized as safe) flavoring agents available or known. As noted above, sweetening agents, sugar substitutes, and high intensity sweeteners and derivatives of these can be used, including but not limited to sucralose, aspartame, acesulfame potassium, saccharine, cyclamate, glycyrrhizine, dihydrochalcones, stevisoide, thaumatin, monellin, neohesperidine, and any of the polyol compounds. A preferred polyols or sugar alcohols are erythritol and maltitol, but any others can be selected, alone or in any of various combinations possible, from, for example, mannitol, sorbitol, and xylitol. Sugar substitutes, as known and available in the art, can also be used, alone or in various combinations. In addition, combinations with other food ingredients, extracts, or supplements can be used, including combinations with one or more extracts, supplements, or ingredients selected from: guarana; taurine; citrus fruits; berries; orange; lemon; lime; tangerine; mandarin; grapefruit; acerola; grape; pear; passion fruit; pineapple; banana; apple; cranberry; cherry; raspberry; chokeberry; grapeseed; peach; plum; grape; currant; black currant; cranberry; blackberry; blueberry; pomegranate; acai; noni; elderberry; goji berry; rosehips; bilberry; hawthorne berry; ginko; gotu kola; rooibos; boysenberry; catuaba; horny goat weed; yohimbe; damiana; red raspberry leaf; vitex berry; blessed thistle; wolfberry; strawberry; mirabelle; watermelon; honeydew; cantaloupe; mango; papaya; botanical flavors derived from cola; tea; white tea; green tea; coffee; vanilla; almond; vegetables; tomato; cabbage; celery; cucumber; spinach; carrot; lettuce; watercress; dandelion; rhubarb; beet; cocona; guava; and han guo.

Any of the aspects or embodiments of the invention can comprise or include formulations of cocoa extract, cocoa powder, or cocoa-containing products that are tablets, gelatin-coated capsules, suspensions, parenteral solutions, ready-to-drink mixes and beverages, powdered beverage mixes, savory condiments, yogurt-based beverages or mixes, milk-based beverages or mixes, soy milk-based beverages or mixes, sugar-free confections, sugar-free chocolate, and/or sugar-free baked goods. A preferred example is a bar product or health bar comprising one or more non-nutritive sweeteners or sugar alcohols. The cacao or cocoa solids or cacao or cocoa mass content of the compositions can generally be equated to a percentage of other cocoa products, such as percent cocoa powder. For example, a cocoa powder can have between about 10% to 25% fat, or more typically 10% to 12% fat, where the fat is generally cocoa butter. Thus, a composition with 85% cocoa powder can be equivalent to a cocoa product with about contain about 60% to about 77% cacao solids or cacao mass. The preferred 85% natural cocoa powder having about 10-12% cocoa butter will thus be equivalent to about 77% to about 74% cacao solids or cacao mass. As noted above, the cocoa powder used can also be processed from extraction methods, where substantially all of the fat is removed.

The examples below present only some of the possible embodiments of the invention and demonstrate only some of the advantages and uses of the composition and methods. The content of the Examples should not be taken as a limitation of the scope of the invention in general.

### EXAMPLE 1

### Sugar-free Cocoa Compositions

A set of human studies is conducted to investigate the effects that various compositions of chocolate have on vascular endothelial function. Compositions consisting of sugar-free chocolate, sugared chocolate, and a placebo with no cocoa are administered to 45 healthy individuals with a body mass index of between 25-35 kg/m². The effects of the various compositions on endothelial function are evaluated by measuring blood pressure (both systolic and diastolic), and flow mediated dilation (FMD) and stimulus-adjusted response measure (SARM).

Flow mediated dilation is a main measure of this study and is assessed by the use of ultrasound technology to measure the diameter of blood vessels—in this case, the brachial artery (main artery in the arm). This technique is known to those of skill in the art and is described in a number of peer-reviewed studies, including those listed and incorporated by reference into this specification. A blood pressure cuff is used to cut-off circulation to the arm and then released. The ultrasound provides a picture of the extent of dilation (diameter) of the artery one minute after the cuff is removed. This measure is taken as a baseline (before eating the chocolate or cocoa composition) and then 2 hours after eating the chocolate or cocoa. It is known by those of skill in the art that the flavanols in chocolate are maximally absorbed at around 2 hours after consumption. Comparing the baseline to the 2 hour measure provides a percent change in the artery's function. The higher the percent change the more the administration of cocoa provides benefits to the artery and blood flow, and thus to cardiovascular health or prophylaxis.

In order to standardize this measure, the concept of stimulus-adjusted response measure (SARM) is used. To calculate SARM, FMD is divided by the change in blood flow at 15 seconds after the cuff is deflated. Systolic and diastolic blood pressure readings are also recorded. Table 1 describes the baseline results with 44 measurements (n=44) in each group of subjects in the sugar-free cocoa group, the +sugar cocoa group, and the placebo group.

TABLE 1
Baseline Values for Each Subject Group -Acute Phase

| | Sugar Free Cocoa | | Placebo |
|---|---|---|---|
| | (n = 44) | +Sugar Cocoa | (n = 44) |
| Variable | Mean ± SD | (n = 44) | Mean ± SD |
| Diastolic Blood | 68.24 ± 13.84 | 67.74 ± 12.62 | 68.91 ± 12.65 |
| Pressure (mmHg) | | | |
| Systolic Blood | 123.38 ± 15.16 | 121.44 ± 15.45 | 121.30 ± 13.89 |
| Pressure (mmHg) | | | |
| Flow Mediated | 5.58 ± 2.62 | 5.84 ± 2.17 | 6.31 ± 2.89 |
| Dilation or FMD (%) | | | |
| Stimulus Adjusted | 0.055 ± 0.045 | 0.055 ± 0.029 | 0.062 ± 0.076 |
| Response Measure or | | | |
| SARM | | | |
| Weight (lbs) | 180.49 ± 22.48 | 179.78 ± 23.32 | 179.84 ± 23.17 |
| Room Temperature | 72.58 ± 0.81 | 72.68 ± 0.83 | 72.60 ± 0.81 |
| (° F.) | | | |
| Age (years) | 52.78 ± 11.03 | 52.78 ± 11.03 | 52.78 ± 11.03 |

SD = Standard deviation;
In all variables, no significance (p-value > 0.05) difference observed between group (one-way ANOVA)

TABLE 2
Exemplary Sugar Free Cocoa Formula

| Ingredient | Percent | Grams/Serving |
|---|---|---|
| Vanillin, USP Grade | 1.88% | 0.22 |
| Ace-K | 0.33% | 0.04 |
| Aspartame | 0.78% | 0.09 |
| Salt | 1.19% | 0.14 |
| Carrageenan, Lact MV-306 | 1.96% | 0.23 |
| Cocoa Powder | 93.86% | 11.00 |
| Total | 100.00 | 11.72 |

TABLE 3A
+Sugar Cocoa Control Formulation

| Sugar Cocoa | Gram/serving | Percent (dry basis) |
|---|---|---|
| Sugar | 45.29 | 78.87% |
| Vanillin, USP Grade | 0.26 | 0.45% |
| Salt | 0.65 | 1.13% |
| Carrageenan | 0.23 | 0.40% |
| Natural Cocoa Powder | 11.00 | 19.15% |
| Total | 57.43 | 100.00% |

TABLE 3B
No Cocoa Polyphenol Control (Placebo)

| | Gram per | |
|---|---|---|
| Placebo (No Cocoa Powder) | serving | Percent (dry basis) |
| Powdered Artificial Vanilla Flavor | 0.05 | 0.08% |
| Powdered Cocoa Creamer | 3.76 | 6.00% |
| Non-Fat Dried Milk | 2.51 | 4.00% |
| Sugar | 49.41 | 78.87% |
| Vanillin, USP Grade | 0.13 | 0.20% |
| Salt | 0.19 | 0.30% |
| Sweet Whey Powder | 6.61 | 10.55% |
| Total | 62.65 | 100.00% |

In the above, one serving is mixed with 8 oz. hot water.

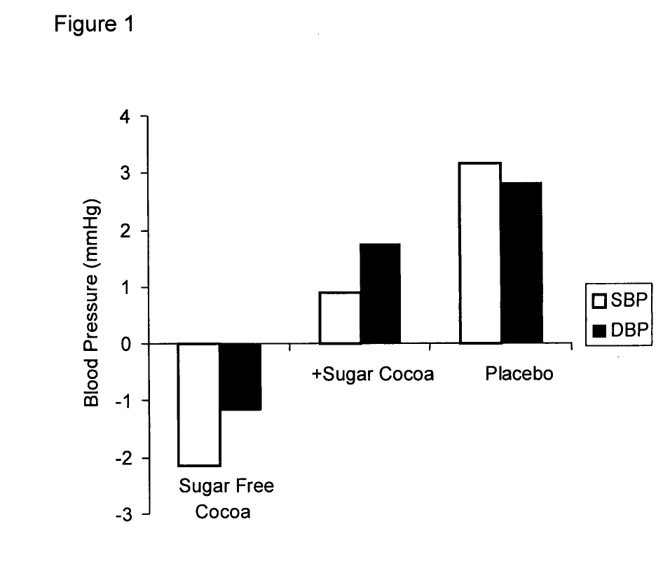

Table 4 reports the changes in the baseline measured 2 hours after the consumption of the interventional beverage. These results are also summarized graphically in FIGS. 1-3.

TABLE 4
Change in Outcome Measures from Baseline after Treatment - Acute Phase

| | Sugar Free Cocoa | | Sugar Cocoa | | Placebo | |
|---|---|---|---|---|---|---|
| | (n = 44) | | (n = 44) | | (n=44) | |
| Variable | Mean ± SD | p-value | Mean ± SD | p-value | Mean± SD | p-value |
| Systolic Blood | −2.14 ± 6.95† | 0.0524 | 0.88 ± 8.72 | 0.5165 | 3.16 ± 5.61 | 0.0006 |
| Pressure (mmHg) | | | | | | |
| Diastolic Blood | −1.17 ± 8.69† | 0.3892 | 1.74 ± 6.20 | 0.0766 | 2.84 ± 5.62 | 0.0019 |
| Pressure (mmHg) | | | | | | |
| Flow Mediated | 5.70 ± 2.64†‡ | <.0001 | 2.03 ± 1.82† | <.0001 | −1.49 ± 2.80‡ | 0.0012 |
| Dilation or FMD | | | | | | |
| (%) | | | | | | |
| Stimulus Adjusted | 0.041 ± 0.047‡ | <.0001 | 0.024 ± 0.048† | 0.0065 | −0.020 ± 0.075‡ | 0.1114 |
| Response Measure or | | | | | | |
| SARM | | | | | | |

In Table 4:
†indicate significance (p-value < 0.05) difference compared to placebo;
‡significance (p-value < 0.05) difference compared to Sugar Cocoa;
SD = Standard deviation;
p-values obtained from paired ttest

Sugar-free cocoa and sugared cocoa ingestion improves endothelial function significantly and lowers blood pressure in the participants, all of whom were slightly overweight to modest obesity (BMI=25-35 kg/m²). While the placebo (cocoa with no flavanols) shows a modest impairment in this measure.

Sugar-free cocoa also shows a statistical trend toward lowering systolic blood pressure (−2.14 mmHg), while there is no effect on diastolic blood pressure. The sugar cocoa does not effect either measure of blood pressure and the placebo worsens blood pressure. (See FIG. 1)

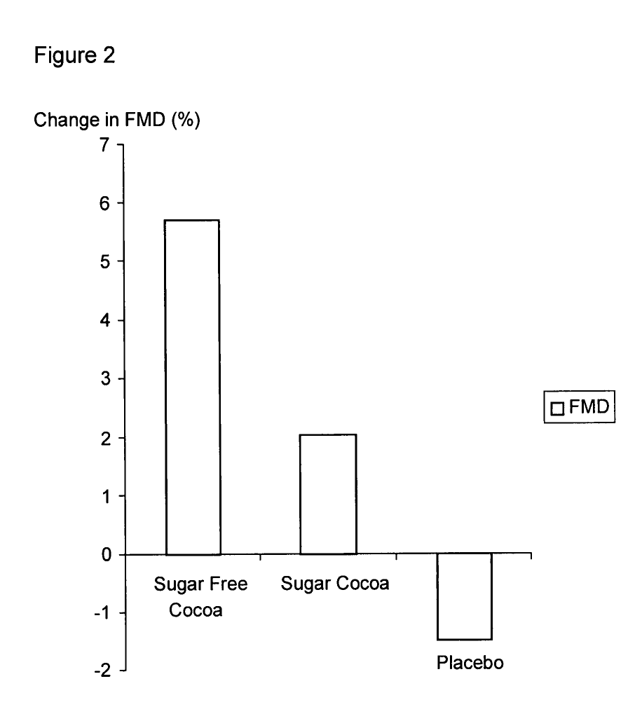

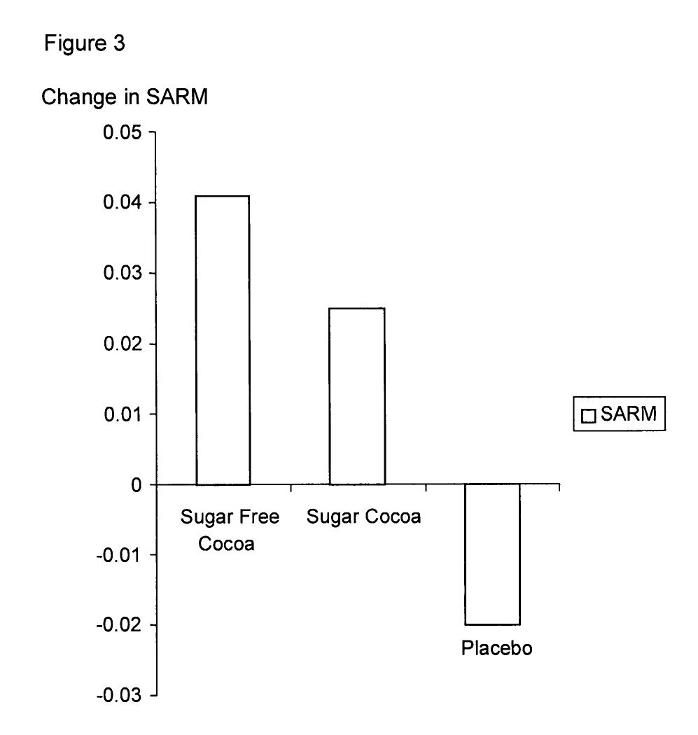

After drinking two cups of sugar-free cocoa, FMD improves (5.7%±2.64) significantly (p<0.0001) from baseline values while the sugar cocoa improves FMD by 2.03% (±1.82) (See FIG. 2). SARM also improves significantly for sugar-free cocoa (0.041±0.047) and for sugar cocoa (0.024±0.048) (See FIG. 3). The placebo worsens FMD by 1.49%±2.80.

In summary, the results show that a sugar-free cocoa composition of the invention has an even greater improvement in arterial function than the previously known results for dark chocolate, while the +sugar cocoa showed an improvement slightly less than that seen with dark chocolate.

Treatment regimens for the cocoa formulations and methods of the invention can vary.

However, preferred administration treatments include: the equivalent of about 10-20 grams cocoa powder twice a day; equivalent of about 20-30 grams cocoa powder twice a day; equivalent of about 30-40 grams natural cocoa powder one or twice a day;

equivalent of about 40-50 grams natural cocoa powder once or twice a day; equivalent of 10 grams of natural cocoa powder two or more times a day. In each case, the preferred administration method would avoid, or substantially avoid the administration of sugar, lactose, glucose, sucrose, or nutritive sugar within about 10-30 minutes before administering cocoa; within about 2 hours after administering cocoa; both within 10-30 minutes before and about 2 hours after administering cocoa; about 90 to about 120 minutes after administering cocoa; within about 90 to about 120 minutes before administering cocoa; both within 90-120 minutes before and 90-120 minutes after administering cocoa; and about 2-3 hours after administering cocoa. Additionally, treatment regimens can include taking the cocoa composition once after waking in the morning and once before bed at night. Various other regimens can be devised. A regiment that maintains a low, or substantially lower than average sugar or glucose level in the blood stream for a period of about 1 hour, or 2 hours, or about 3 hours after administering the cocoa is preferred.

Additional embodiments, where less than about 10 grams of natural cocoa powder or the equivalent in cocoa polyphenols, is administered can also be developed. The amount of cocoa or cocoa polyphenols administered to achieve an effective amount for the improvement of FMD or other measurable risk factors can be varied by more frequent administrations, for example. Thus, the invention encompasses methods where more than 10 grams of natural cocoa powder is administered, or the equivalent in cocoa polyphenols, as wells as methods where less than 10 grams is administered. In each case, the number of administrations a day or per time period can be varied to achieve the synergistic results noted here.

### EXAMPLE 2

### Dark Chocolate

A set of human studies is conducted to investigate the effects that dark chocolate compositions have on vascular endothelial function. Compositions consisting of dark chocolate (74 gram bar of dark, solid chocolate equivalent to 22 grams of cocoa powder) are compared to white chocolate control. Each group is administered the dark chocolate or white chocolate after overnight fast and measured for effects on FMD before administration and two hours after administration. After 7 days, a cross-over study where the dark chocolate group is switched with the white chocolate group for another 7 days of the same measurements.

Table 5 is the baseline values for subjects in the cross-over study, and Table 6 provides a summary of the results of the vascular reactivity studies.

TABLE 5
Demographic characteristics and baseline values

| | Dark Chocolate | Placebo | |
|---|---|---|---|
| | (n = 45) | (n = 45) | |
| Variable | Mean ± SD | Mean ± SD | p-value |
| Age (years) | 52.78 ± 11.03 | 52.78 ± 11.03 | 1.0000 |
| Weight (lbs) | 179.11 ± 22.23 | 178.84 ± 22.42 | 0.9549 |
| Body Mass Index | 30.14 ± 3.31 | 30.09 ± 3.26 | 0.9396 |
| (kg/m²) | | | |
| Room Temperature | 72.00 ± 0.21 | 72.04 ± 0.67 | 0.6744 |
| (° F.) | | | |
| Diastolic Blood | 68.64 ± 11.77 | 69.87 ± 11.66 | 0.6218 |
| Pressure (mmHg) | | | |
| Systolic Blood | 124.80 ± 16.95 | 122.82 ± 15.18 | 0.5613 |
| Pressure (mmHg) | | | |
| Flow Mediated | 7.36 ± 3.55 | 9.10 ± 4.82 | 0.0553 |
| Dilation or FMD (%) | | | |
| Stimulus Adjusted | 0.08 ± 0.08 | 0.07 ± 0.17 | 0.6319 |
| Response Measure or | | | |
| SARM* | | | |

*indicate occasional missing;
SD = Standard deviation;
p-values obtained from student ttest

TABLE 6
Change in Outcome Measurements from Baseline

| | Dark Chocolate (n = 45) | | Placebo (n = 45) | |
|---|---|---|---|---|
| Variable | Mean ± SD | p-value | Mean ± SD | p-value |
| Systolic Blood | −3.24 ± 5.82 | 0.0005 | 2.68 ± 6.64 | 0.0104 |
| Pressure (mmHg)† | | | | |
| Diastolic Blood | −1.40 ± 3.91 | 0.0206 | 2.73 ± 6.36 | 0.0067 |
| Pressure (mmHg)† | | | | |
| Flow Mediated | 4.28 ± 3.43 | <.0001 | −1.84 ± 3.33 | 0.0007 |
| Dilation or FMD | | | | |
| (%)† | | | | |
| Stimulus Adjusted | 0.04 ± 0.10 | 0.0129 | 0.01 ± 0.18 | 0.7902 |
| Response Measure | | | | |
| or SARM* | | | | |

†indicate significant (p-value < 0.05) difference between treatment groups;
SD = Standard deviation;
*indicate occasional missing;
p-values obtained from paired ttest

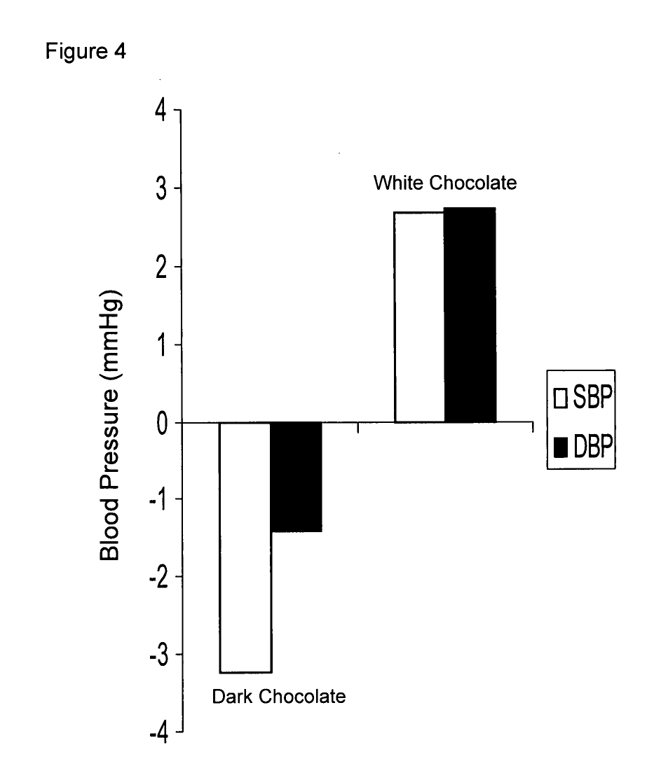

Dark chocolate ingestion improves endothelial function and lowers blood pressure in individuals with a BMI between 25 and 35 kg/m² (Table 6). After a single dose ingestion of dark chocolate, FMD improves (4.28±3.43) significantly (p<0.0001) from baseline. SARM also improves significantly (0.04±0.10; p=0.0129). A single dose ingestion of dark chocolate lowers both systolic (−3.24±5.82; p=0.0005) and diastolic (−1.40±3.91; p=0.0206) blood pressure from baseline (FIG. 4).

Ingestion of the white chocolate placebo shows deterioration in endothelial function and blood pressure for individuals with BMI between 25 and 35 kg/m² (−1.84±3.33; p=0.0007). However SARM did not change significantly (0.01±0.18; p=0.7902). Both systolic and diastolic blood pressure increases (2.68±6.64; p=0.0104 and 2.73±6.36; p=0.0067 respectively) after single dose ingestion of placebo. There is a significant improvement in FMD and blood pressure after single dose ingestion of dark chocolate (p<0.05) compared to single dose ingestion of placebo.

The results here comprise the first clinical trial demonstrating improved endothelial function, as well as decreased systolic and diastolic blood pressure, in adults with elevated BMI following acute consumption of flavonoid-rich and dark chocolate products. In addition, the results here are the first to examine the effects of sugar free cocoa ingestion on these biomarkers of vascular function.

Acute consumption of dark chocolate significantly improves diastolic flow mediated dilation of the brachial artery as compared to placebo. Placebo (white chocolate, which is procyanidin-poor) adversely affected endothelial function after acute ingestion. Without limiting the invention to any particular mode of action or physiological effect, the improvement in FMD noted after dark chocolate consumption is most likely related to the increase in plasma epicatechin concentrations, which increases endothelium-derived vasodilators and thereby improves endothelial function. Dark chocolate consumption also increases Stimulus Adjusted Response Measure (SARM) significantly and decreases systolic blood pressure and diastolic blood pressure. The reduction in systolic blood pressure is greater than in diastolic blood pressure.

Our results are consistent with most studies done on dark chocolate ingestion in several different population groups. Previous studies examining the effect of cocoa ingestion on blood pressure report mixed results.

Acute consumption of sugar-free cocoa compositions shows similar improvements on endothelial function and blood pressure. Previous studies have indicated that postprandial hyperglycemia and acute glycemic load result in inflammatory responses and endothelial dysfunction. The results here corroborate these findings. However, the results here also show that cocoa consumption increases brachial artery flow mediated dilatation in both the sugar-free cocoa and cocoa with sugar group, but the improvement is unexpectedly greater in the sugar-free cocoa group. Sugar-free cocoa reduced systolic and diastolic blood pressure, while ingestion of cocoa with sugar results in increases in systolic and diastolic blood pressure. It appears that procyanidin-rich, sugar-free cocoa attenuates adverse effects of sugar and elicits a cardio protective response.

The study described here have limitations and the results could be influenced by potential confounders, such as the association between nutrient intake and endothelial and physiological responses, including unmeasured or inaccurately measured dietary intake data, changes in physical activity, noncompliance, vasoactive medication use, and genetic factors. However one can control for these confounders through strict eligibility criteria, randomization, and cross-over so that subjects serve as their own controls for testing of the principal hypotheses.

The results described here support a beneficial action of acute consumption of dark chocolate and sugar-free cocoa compositions on cardiovascular status, as evidenced by decreases in systolic and diastolic blood pressures and improvement in endothelial function. Individuals with a BMI between 25-35 kg/m² are considered overweight or obese and are at elevated risk for cardiovascular disease. While more research to assess the sustained effects of dark chocolate and sugar-free cocoa consumption in this population, the improvements demonstrated benefit the test populations.

The examples presented above and the contents of the application define and describe examples of the many cocoa compositions, products, and methods that can be produced or used according to the invention. None of the examples and no part of the description should be taken as a limitation on the scope of the invention as a whole or of the meaning of the following claims.

What is claimed is:

1. A therapeutic or prophylactic method of treating atherosclerosis, thrombosis, heart attack, stroke, or vascular circulation conditions comprising administering to a subject in need thereof a sugar-free composition comprising at least about 10 grams of a formulation of at least 60% natural cocoa solids twice a day.

2. The method of claim 1, wherein the sugar-free composition further comprises a sugar substitute.

3. The method of claim 1, wherein the sugar-free composition further comprises an appetite suppressing compound.

4. The method of claim 1 further comprising removing dietary sugar and sucrose from the diet for about 2 hours after administration of the sugar-free composition.

5. The method of claim 1, wherein the sugar-free composition comprises at least about 80% natural cocoa solids.

6. The method of claim 1, wherein the sugar-free composition comprises about 85-95% natural cocoa solids.

7. The method of claim, 6, wherein the sugar-free composition further comprises a sugar substitute.

8. The method of claim 7, wherein the sugar substitute is a sugar alcohol.

9. The method of claim 1, further comprising maintaining the subject on a diet substantially free of sugar or sucrose for about 1 hour after administration.

10. The method of claim 1, further comprising maintaining the subject on a diet substantially free of sugar or sucrose for about 2 hours after administration.

11. A sugar-free cocoa formulation for improving endothelial function, comprising at least about 85% natural cocoa powder and one or more sugar substitutes.

12. The sugar-free formulation of claim 11, further comprising a flavoring agent.

13. The sugar-free formulation of claim 12, wherein the flavoring agent is vanillin.

14. The sugar-free formulation of claim 13, comprising at least about 90% natural cocoa powder.

15. The sugar-free formulation of claim 15, comprising about 92-95% natural cocoa powder.

16. The sugar-free formulation of claim 11, present as a product selected from one of: tablet; gelatin-coated capsule; suspension; parenteral solution; ready-to-drink mix; beverage; powdered beverage mix; savory condiment; yogurt-based beverage or mix; milk-based beverage or mix; soy milk-based beverage or mix; sugar-free confection; sugar-free chocolate or chocolate coating; and sugar-free baked good.

17. The sugar-free formulation of claim 14, present as a product selected from one of: tablet; gelatin-coated capsule; suspension; parenteral solution; ready-to-drink mix; beverage; powdered beverage mix; savory condiment; yogurt-based beverage or mix; milk-based beverage or mix; soy milk-based beverage or mix; sugar-free confection; sugar-free chocolate or chocolate coating; and sugar-free baked good.

18. The sugar-free formulation of claim 17, wherein the sugar substitute is one of more of a sugar alcohol, ace-K, and aspartame.

19. A method of evaluating a diet that includes a sugar-free cocoa composition for effecting at least one cardiovascular risk factor, comprising orally administering to a human subject at least about 10 grams of a composition comprising at least 85% natural cocoa powder, the composition also being substantially free of sucrose or nutritive sweetener; maintaining a diet substantially free of sucrose or nutritive sweetener for at least about 1 hour after administration; and measuring a cardiovascular risk factor in the subject after administration.

20. The method of claim 19, wherein the measurement is by FMD.

21. The method of claim 19, wherein the subject is administered 10 grams of the composition twice a day.

22. The method of claim 20, wherein the subject is administered 10 grams of the composition twice a day

23. The method of claim 21, wherein the composition comprises about 90-95% natural cocoa powder.

24. A method of testing the effect of a sugar-free composition on blood pressure in a human subject, comprising orally administering to the human subject at least about 10 grams of a composition comprising at least 85% natural cocoa powder, the composition also being substantially free of sucrose or nutritive sweetener; maintaining a diet substantially free of sucrose or nutritive sweetener for at least about 1 hour after administration; and measuring the blood pressure in the subject after administration.

25. A method of testing the effect of a sugar-free composition on the measurable conditions or status of any one or more an individual's risk for adverse atherosclerosis, thrombosis, heart attack, stroke, or vascular circulation conditions, comprising orally administering to the individual at least about 10 grams of a composition comprising at least 85% natural cocoa powder, the composition also being substantially free of sucrose or nutritive sweetener; maintaining a diet substantially free of sucrose or nutritive sweetener for at least about 1 to 2 hours after administration; and measuring the status one or more of endothelial function by FMD, LDL levels, HDL levels, triglyceride levels, lipoprotein A levels, or total cholesterol levels after administration.

26. The method of claim 25, wherein the testing is repeated.

27. The method of claim 26, wherein the testing is repeated on a substantially daily basis for a period of about two weeks to about two months.
